(19) United States
(12) Patent Application Publication
(10) Pub. No.: US 2012/0004493 A1
(43) Pub. Date: Jan. 5, 2012

(54) SYSTEM FOR DELIVERING TREATMENT AGENTS

(76) Inventors: Troy S. Hedger, Rancho Cucamonga, CA (US); Bobby Alan Robnett, Anaheim, CA (US); Carlton L. Tunnell, JR., Temecula, CA (US); James A. Wilson, Follansbee, WV (US)

(21) Appl. No.: 12/830,083

(22) Filed: Jul. 2, 2010

(51) Int. Cl. A61M 36/06; B23P 11/00; B23K 31/02

(52) U.S. Cl. 600/4; 228/176; 29/428

(57) ABSTRACT

A system for delivering a treatment agent such as a radioactive source to a targeted treatment site within a patient's body is disclosed. Aspects of the device provided herein include a cable with contiguous sections having different flexibility characteristics made from a same material or material mixture throughout. For example, the material can mixture have first filaments made from a first material and second filaments made from a second material wherein the first material and second material having different annealing temperatures.

### BACKGROUND

Treatment agents often need to be placed at or adjacent to a site requiring treatment within a patient's body. For example, brachytherapy is a form of radiotherapy wherein a radioactive source is placed at or adjacent to a site requiring radioactive treatment within a patient's body. Brachytherapy has become increasingly important in the treatment of certain diseases, especially cancer, in that the radioactive source can be targeted to localized areas within the body to ensure effective treatment of the affected site while minimizing the risk of unnecessary damage to healthy neighboring tissues or organs.

The radioactive source is normally contained within a well-insulated source housing. The source housing is attached to a drive member or a guide wire, usually a flexible cable, which will guide the radioactive source to a targeted treatment site within the patient's body. To this end, a tubular guide, such as a hollow needle, a flexible tube or a catheter, is first placed at the targeted treatment site. Once the tubular guide is in place, the source housing, driven by the guide wire, follows the path provided by the tubular guide to the targeted treatment site. When the targeted treatment site is in an area that is not readily accessible, the path that the tubular guide traverses to reach the targeted treatment site may comprise many turns, tight angles and/or curves. The source housing, made of metal, has very limited flexibility. Thus, the guide wire needs to be solid and sturdy to propel the source housing through the tubular guide yet sufficiently flexible to guide it through curves without kinking.

The attempt to develop a guidewire with desired flexibility and sturdiness is described in U.S. Pat. No. 6,196,964. However, the structure of this patent requires use of a separate adaptor between the guidwire and capsule holding a radioactive source. It is desirable to have a device without the complexities added by the adaptor, and the risk that the connection between the guidewire and adaptor, or the connection between the adaptor and the capsule of the radioactive source and fail in use.

### SUMMARY

An exemplary system for the delivery of a treatment agent to a treatment site of a patient's body is discussed herein. The system according to the present invention includes a medical device for delivering a treatment agent to a site requiring treatment. The device comprises a source housing comprising an interior cavity for receiving a treatment agent disposed therein and a cable designed for placement of the source housing. The cable is comprised of first and second sections. The first section is connected to the housing and is closer to the housing than is the second section. The first section is formed from a first material and the second section is formed from a second material. The flexibility of the first section is greater than the flexibility of the second section due to heat treatment of at least one of the first and second materials. Preferably, for ease of manufacture, before heat treatment the first and second materials are the same, and can be the same material mixture, where the mixture comprises at least two different materials.

In one example of a system having features of the present invention, the source housing comprises an interior cavity having a radioactive source disposed therein. A cable comprising a first section and a second section made from a same material mixture throughout the first section and the second section is used. The first section has a flexibility greater than that of the second section.

Another feature discussed herein is a guide wire for the delivery of a radioactive source to a treatment site of a patient's body. In one example, a guide wire is described comprising an elongated cable comprising a first section and a second section made from a same material mixture throughout the first section and the second section. The device wherein the material mixture comprises a plurality of first filaments and a plurality of second filaments, the first filaments made from a first material and the second filaments made from a second material. The device wherein the first material has a first annealing temperature and the second material has a second annealing temperature, which is higher than the first annealing temperature.

In yet another example, a method for forming a medical device for delivering a radioactive source to a targeted treatment site is described. The method can comprise the steps of providing a source housing comprising a radioactive source disposed therein and providing a cable comprising a first section and a second section made from a same material mixture throughout the first and second sections. The material mixture comprises a plurality of first filaments and a plurality of second filaments, the plurality of first filaments made from a first material and the plurality of second filaments made from a second material. The first material has a first annealing temperature and the second material has a second annealing temperature, which is higher than the annealing temperature of the first material. The method further comprising the steps of annealing the first section to at least the first annealing temperature and connecting the source housing to the cable.

Consequently, the first section becomes more flexible relative to the second section, although both the first and second sections are formed from the same identical material(s) throughout and have the same starting flexibility characteristics. As such, a further aspect of the present device, system, and method may be understood to include a cable having a first state and a second state and wherein a section of the cable is more flexible in the second state than when in the first state.

In a further example, a multi-strand working cable is described comprising a first state in which the entire cable comprises a first flexibility and a second state in which the same working cable comprises the first flexibility and a second flexibility, which is greater than the first flexibility. The cable also comprises a more flexible section in the second state than for the same section when in the first state. For example, the first section of the cable is more flexible in the second state than when in the first state.

In an exemplary embodiment, several factors, including material selections, thickness of the filaments and relative quantities of the first versus second filaments, can be manipulated to obtain a desired flexibility for the various sections of a cable for use in brachytherapy. Thus, a further aspect of the present method is understood to include a method for regulating the flexibilities of the first and second sections of a guide wire by varying the material and/or thickness and/or quantity of the first and second filaments that make up the cable.

### DRAWINGS

The various embodiments of the present delivery device, systems, and associated methods now will be discussed in detail with an emphasis on highlighting the advantageous features. These embodiments depict the novel and non-obvious delivery device shown in the accompanying drawings, which are for illustrative purposes only. These drawings include the following figures, in which like numerals indicate like parts:

FIG. 1 is a side view of a medical device for delivering a radioactive source to a treatment site; and

FIG. 2 is a cross-sectional end view of the cable or guide wire of the medical device of FIG. 1 taken along line 2-2.

### DESCRIPTION

The detailed description set forth below in connection with the appended drawings is intended as a description of the presently preferred embodiments of devices and methods related to the delivery of a radioactive source to a treatment site within a patient's body and are not intended to represent the only forms in which the present invention may be constructed or utilized. The description sets forth the features and the steps for constructing and using the device in connection with the illustrated embodiments. It is to be understood, however, that the same or equivalent functions and structures may be accomplished by different embodiments that are also intended to be encompassed within the spirit and scope of the invention. As denoted elsewhere herein, like element numbers are intended to indicate like or similar elements or features.

As used herein, the terms “first”, “second”, “proximal”, and “distal” are meant to distinguish different items, sections, or locations only for similar features or structures and for reference purposes but not to limit their scope. For example, a first material and a second material can be reversed by changing one's perspective without changing the scope of the two materials, unless the context indicates otherwise. The source housing can be connected to the cable before or after heat treatment such as by annealing. As used herein, the term “flexible” means capable of being bent to flex, such as being bent repeatedly without injury or damage.

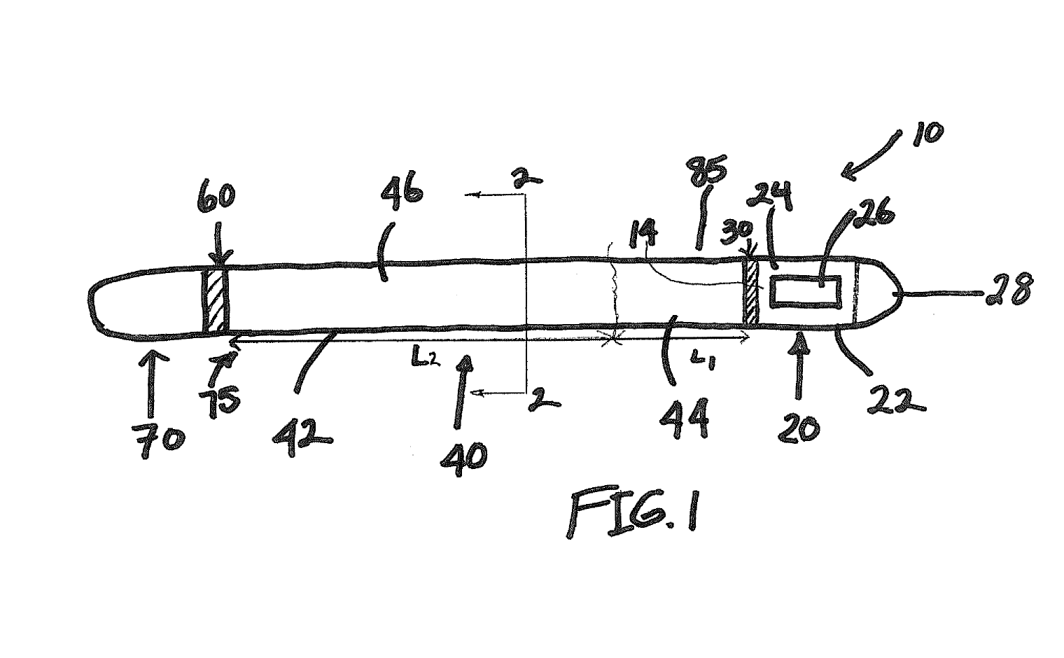

FIG. 1 illustrates an embodiment of a medical device or apparatus 10 for delivering a radioactive source to a treatment site within a patient's body. In one exemplary embodiment, the medical device 10 comprises a source housing 20 connected to a guide wire 40. The source housing 20 is conventionally made from a solid and neutral metal, such as stainless steel, titanium, platinum, etc. In other embodiments, the source housing 20 is made from two or more materials, such as from an outer layer of stainless steel or other suitable metals and an inner layer of titanium or platinum. In alternative embodiments, the source housing 20 is made from a composite, such as metal alloys.

In one embodiment, the source housing 20 comprises a tubular body 22 having a cavity 24 for receiving a radioactive source 26. The radioactive source 26 includes naturally occurring radioactive materials, such as radium-226, or isotopes produced from neutral activation or nuclear fission, such as Iridium-192, Palladium-103 or Ytrium-90. In alternative embodiments, the radioactive source 26 comprises a mixture of radioactive materials. The radioactive source 26 is encapsulated in the cavity 24 of the source housing 20 for use to deliver to the treatment site. In one embodiment, the cavity 24 is sealed at its front end by a plug or cap 28, which is attached to the body 22 using conventional means, such as welding. In one example, the housing is formed with an integral plug 28 and the source housing is placed into the interior cavity via the rear opening 14, which is also used to attach the source housing 20 to the guide wire 40. The attachment can be referred to as a connecting means 30. In a specific embodiment, the connecting means 30 is a weld. In alternative embodiments, the connecting means 30 includes an interference fit wherein an end of the guide wire 40 is structured to project into the opening end 14 of the tubular body 22, such as by way of a simple snap fit arrangement or by crimping the open end.

Referring again to FIG. 1, the guide wire 40 comprises an elongate cable 42. In one embodiment, the cable 42 has a cylindrical shape cross-section. However, other appropriately shaped tube configurations are also suitable. The cable 42 is attached to the source housing 20 via the connecting means 30 at the distal end 85 of the cable. The cable 42 comprises a linking member 60 at the proximal end 75 for connecting the guide wire 40 to a coupling element 70, which enables the guide wire 40 to maneuver the source housing 20 through a tubular guide, such as a catheter (not shown). In one embodiment, the linking member 60 comprises a weld. In alternative embodiments, the coupling element 70 comprises a linking end (not shown) which is dimensioned or structured to engage in an interference fit or friction fit with an opposing end of the guide wire 40.

In one embodiment, the cable 42 comprises a plurality of contiguous sections unitarily formed from a same material or material mixture extending between the proximal end 75 and the distal end 85. As used herein, unitarily formed is understood to mean the same throughout or monolithically formed between the first section and the second section in a longitudinal direction. Along a cross-sectional direction, the first and second sections may have a single large gauge cable or a plurality of strands forming a cable with each cable or strand being unitarily formed in the longitudinal direction. In one embodiment, the cable 42, which is formed from the same uninterrupted material(s) from the proximal end 75 to the distal end 85, comprises at least two sections having different flexibility characteristics. In another embodiment, the cable 42 comprises a plurality of sections having different flexibility characteristics, such as three or more sections with different flexibility characteristics. In one example, the cable 42 from the proximal end 75 to the distal end 85, and up to the connecting means 30 that joins the source housing 20 to the guide wire 40, does not incorporate a weld or any attachment means. In another example, the guide wire 40, without any attachment means between the proximal end 75 and the distal end 85, comprises at least two sections having different flexibility characteristics.

Referring again to FIG. 1, the cable 42 comprises a first section 44 adjacent the connecting means 30 (i.e., near the distal end 85) and a second section 46, which may be considered any other part or section of the cable 42 that does not include or cover the first section 44. For example, the second section 46 can be considered adjacent the first section 44 with no intervening section or sections in between but not necessarily include the remainder of the cable in the proximal direction. As further discussed below, a novel feature of the present device, system, and method is a singularly formed cable between the first section and the second section wherein the two section scan be distinguished from one another only by flexibility characteristics, which may be a finite change in characteristic or a gradual change in characteristic. The first section 44 and the second section 46 have respective lengths L1 and L2, with L2 being greater than L1. In one embodiment, the length L2 is at least two times the length L1, such as four to six times longer than L1. In other embodiments, L2 is at least ten times longer than L1.

In a specific embodiment, the first section 44 and the second section 46, unitarily or singularly formed from a same material or material mixture, have different flexibility characteristics. In a preferred embodiment, the first section 44 has a greater flexibility than that of the second section 46. In another embodiment, alternating flexible and less flexible sections extend the whole length of the guide wire of the second section 46 to the proximal end 75. In use, the stiffer and longer second section 46 provides the guide wire 40 with sufficient rigidity to push the source housing 20 through the tubular catheter and the more flexible first section 44 allows the source housing 20 to readily move through the bends and curves of the tubular guide.

In one embodiment, the cable 42 is singularly formed from a single strand cable, i.e., a large gauge wire. In another embodiment, the cable 42 is made from a plurality of filaments or strands. In one embodiment, the cable 42 comprises a mixture of filaments made from at least two different materials. In a specific embodiment, the cable 42 comprises a mixture of filaments made from three or more different materials. The different flexibility characteristics may be formed by treating the cable after forming it, as further discussed below.

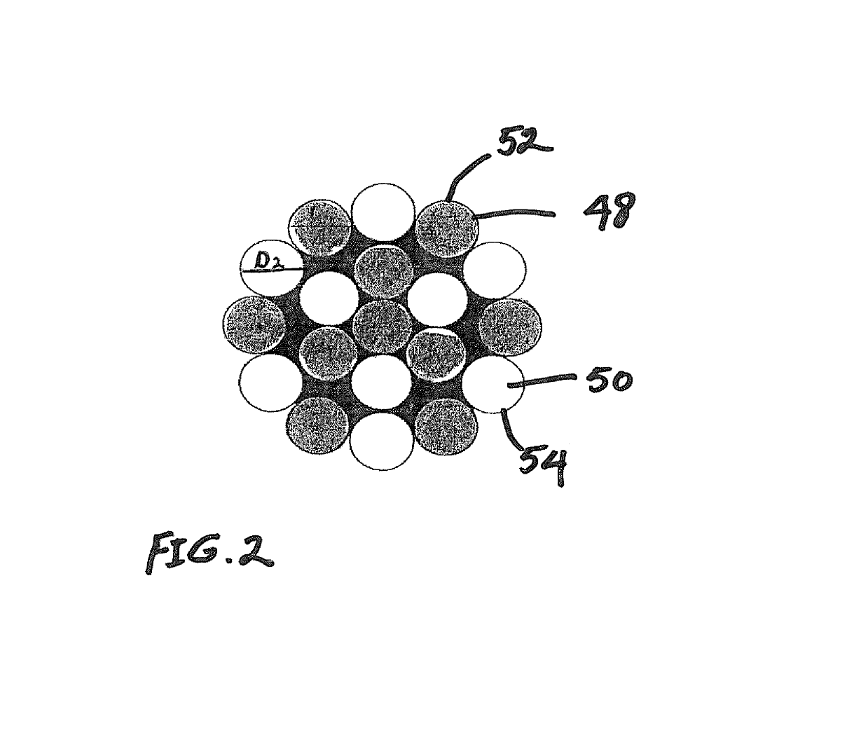

FIG. 2 is a cross-sectional end view of the cable 42 of FIG. 1. As shown in FIG. 2, the cable 42 comprises a mixture of first filaments 48 and second filaments 50. The first filaments 48, having a first diameter D1, are made from a first material 52. For example, the first material can include, but not limited to, any suitable metals and/or metal alloys. The second filaments 50, having a second diameter D2, are made from a second material 54. For example, the second material may include, but not limited to, any suitable metals and/or metal alloys. In one example, D1 equals D2. In another example, D1 is less than D2. In still another example, D1 is greater than D2. Preferably, the first material 52 has an annealing temperature different from that of the second material 54. As used herein, annealing temperature is understood to mean the temperature required to induce ductility of a material or to soften it. In a preferred embodiment, the first material 52 has an annealing temperature that is lower than the annealing temperature of the second material 54. Thus, when a section of the cable 42, such as for example the first section 44, is subjected to the lower annealing temperature of the first material 52, the first filaments 48 anneal and become softer while the physical characteristics of the second filaments 50 remain the same. Consequently, the first section 44 becomes more flexible relative to the second section 46 although both the first and second sections are formed from the same identical material(s) throughout and have the same starting flexibility characteristics. As such, aspect of the present device, system, and method may be understood to include a cable having a first state and a second state and wherein a section of the cable is more flexible in the second state than when in the first state.

The cable 42 of the instant medical device comprises contiguous first and second sections 44 and 46 that are unitarily made from the same material mixture, such as a mixture of first filaments 48 and second filaments 50, but have different flexibility characteristics. By material mixture, the cable is understood to mean a single material mixture, such as NiTi, or two or more mixtures, which include strands or filaments made from different materials, such as SS, NiTi, and Copper or a different shaped-memory alloy. As described, the first section 44 has a flexibility characteristic that is greater than that of the second section 46. In another embodiment, the cable 42 is made from a single wire that has different flexibility sections, which may be made by annealing one section of the single wire and not the other.

In one embodiment, electrical resistance, thermal radiation, or other known conventional heating means may be used to anneal the section or sections of the cable to be annealed. The annealing process may further include cooling the adjacent section of the section to be annealed. For example, a coolant, such as a cool gas stream or a liquid, may be used to keep the section adjacent the section to be annealed cool to prevent unwanted annealing.

Thus, an aspect of the present assembly and method is understood to include a multi-strand cable comprising strands made from at least two different materials, which are the same from a distal end and outside of the distal end, such as towards the proximal end. For easy reference, the cable having the different properties may be referred to as a “working cable”. The multi-strand working cable comprises a first state in which the entire cable comprises a first flexibility and a second state in which the same working cable comprises the first flexibility and a second flexibility, which is greater than the first flexibility. The cable also comprises a more flexible section in the second state than for the same section when in the first state. For example, the first section 44 of the cable is more flexible in the second state than when in the first state.

In a specific embodiment, the cable 42 comprises a mixture of first filaments 48 made from a metal alloy, such Nickel Titanium or NiTi, and second filaments 50, made from a high tensile strength material, such as stainless steel. In one embodiment, the number of strands of first filaments 48 is the same as the number of strands of second filaments 50. In another embodiment, the number of first filaments 48 is less than the number of second filaments. In still yet another embodiment, the number of first filaments 48 is greater than the number of second filaments. Stainless steel has an annealing temperature above 1040° C. versus an annealing temperature of about 400° C. for NiTi.

Referring again to FIG. 1, when the first section 44 is subjected to an annealing procedure at 400° C., the first NiTi filaments 48 of the first section 44 anneal and become softer, rendering the first section 44 more flexible than in its pre-annealing state and more flexible than the second section 46. The stiffness of the second section 46 and the stiffness of the second filaments 50 remain generally the same or constant between the first state and the second state, which is understood to mean a pre-treated state and a post-treated state, respectively. Thus, because the first filaments of the first section 44 have been annealed when in the second state, the flexibility of the first section 44 is greater than that of the second section 46 as it contains annealed first filaments 48.

Thus, aspects of the present device and method are understood to include a guide wire for delivering a radioactive source to a targeted treatment site, the guide wire comprising contiguous first and second sections, unitarily formed from a same material mixture, and wherein the first and second sections have different flexibility characteristics.

A further aspect of the device and method is understood to include a guide wire for delivering a radioactive source to a targeted treatment site; the guide wire comprising a cable comprising a mixture of first filaments and second filaments. The first filaments are made from a first material and the second filaments are made from a second material, and wherein the first material and the second material have different annealing temperatures.

A further aspect of the present method is understood to include a method for forming a medical device for delivering a radioactive source to a targeted treatment site, said method comprising the step of providing a source housing having a radioactive source positioned therein. The method further comprising the step of providing a cable having contiguous first section and second section, the first section and the second section are unitarily made from a same material mixture, and wherein the first section has a flexibility that is greater than that of the second section. The method further comprising the step of connecting the first section to the source housing.

A further aspect of the present method is understood to include a method for making a guide wire for delivering a radioactive source to a targeted treatment site within a patient's body comprising the step of providing a cable having a first section and a second section, the cable comprising a mixture of first filaments made from a first material and second filaments made from a second material, wherein the first material has a first annealing temperature that is lower than the annealing temperature of the second material. The method further comprising the step of annealing the first section at the first annealing temperature.

The relative degree of flexibility of the first section 44 may be controlled by material selection of the first filaments 48 and/or second filaments 50. In one embodiment, as set forth above, the first filaments 48 are made of NiTi alloy and the second filaments 50 are made of stainless steel. For a NiTi alloy/stainless steel product, the heat treatment can be from about 400 to about 450° C. for about 30 to about 60 minutes followed by a water quench at room temperature. In alternative embodiments, as non-limiting examples, the first filaments 48 may be made from aluminum, copper, nickel, chromium alloys and/or alloys thereof and the second filaments 50 may be made from any suitable metals and/or metal alloys. The lower the annealing temperature of the first material 52 from which the first filaments 48 are made, the more flexible the first filaments 48 can become upon annealing. Thus, a cable comprising first filaments 48 made from aluminum alloys having an annealing temperature in the range of 300-410° C. will have a more flexible first section 44 than a cable comprising first filaments 48 made of copper alloys that have an annealing temperature range of 700-800° C. Thus, the degree of flexibility of the first section 44 and of the second section 46 can be manipulated by material selections of the first and second filaments. Furthermore, use of shaped memory alloys (SMAs), such as NiTi, copper-zinc-aluminum-nickel, and copper-aluminum-nickel, allow for greater bending due to well known pseudo-elasticity properties of SMAs.

Other than material selection, the relative degree of flexibility of the various sections of the cable 42 may be controlled or regulated through, among other factors, the thickness and the number of the first filaments 48 versus the second filaments 50, as further discussed below. Also, different sections of the guide wire may be annealed so that the same guide wire may have more than three different sections of different flexibility characteristics.

Referring again to FIG. 2, in one embodiment, the first filaments 48 have the same diameter as the second filaments 50. In alternative embodiments, the first diameter D1 and the second diameter D2 are different from one another. In a specific embodiment, the first diameter D1 is less than the second diameter D2. In alternative embodiments, the first diameter D1 is greater than the second diameter D2. The smaller the first diameter D1, the softer the first filaments become at a given annealing temperature, the more flexible the first section 44 can become. Thus, the thickness of the first diameter D1 can be manipulated to achieve a targeted flexibility for the first section 44 relative to the second section 46.

Furthermore, in addition to material selection and thickness of the filaments, the relative numbers of first filaments 48 and second filaments 50 may also control the degrees of flexibility of the first and second sections 44, 46 of the cable 42. In one example, the cable 42 comprises a quantity N1 of first filaments 48 and a quantity N2 of second filaments 50. In one embodiment, the quantities N1 and N2 may be the same. In another embodiment, N1 is greater than N2. In another example, N1 is less than N2. In this case, at a given annealing temperature, the higher the number of the first filaments 48, the more flexible the first section 44 can become. Thus, the number of the first filaments 48 relative to the number of the second filaments 50 can be manipulated to obtain a desired flexibility for the first section 44 relative to the second section 46.

In an exemplary embodiment, the above-discussed factors, including material selections, thickness of the filaments and relative quantities of the first and second filaments, can be manipulated to obtain a desired flexibility for the various sections of the cable 42. Thus, a further aspect of the present method is understood to include a method for regulating the flexibilities of the first and second sections of the guide wire by varying the material and/or thickness and/or quantity of the first and second filaments.

Many alterations and modifications may be made by those having ordinary skill in the art, without departing from the spirit and scope of the invention. For example, rather than making a cable from a mixture of materials, a single material whose flexibility can be either increase or decreased by treatment, such as by heat treatment, can be used. If treatment increases flexibility, then the first section is heat treated. If treatment decreases flexibility, then the second section is treated. In addition, the cable need not be made with two types of filaments, but can be made with a filament surrounded or impregnated with a material, such as a polymerizable plastic, wherein treatment can change flexibility of any of the materials used for making the cable. Moreover, the invention is not limited to heat treatment, but can be used with any processing that can be used to change flexibility, such as cold treatment, a chemical processes such as acid wash or polymerization process initiated by heating a catalyst or use of UV light, or light of other wavelength. Also, although the present invention has been described with respect to using radioactive sources for patient treatment, other treatment agents can be used, such as drugs and other therapeutic agents. Therefore, it must be understood that the illustrated embodiments have been set forth only for the purposes of examples, and that the embodiments should not be taken as limiting the invention as defined by the following claims. The following claims are, therefore, to be read to include not only the combination of elements which are literally set forth, but all equivalent elements for performing substantially the same function in substantially the same way to obtain substantially the same result. The claims are thus to be understood to include those that have been illustrated and described above, those that are conceptually equivalent, and those that incorporate the ideas of the invention.

What is claimed is:

1. A medical device for delivering a treatment agent to a site requiring treatment, the device comprising:
a source housing comprising an interior cavity for receiving a treatment agent disposed therein; and
a cable comprising a first section and a second section made from the same material mixture throughout the first section and the second section, the mixture comprising at least two different materials;
wherein the source housing is attached to the first section; and
wherein the first section has a flexibility greater than that of the second section.

2. The medical device of claim 1, wherein the first section has a first diameter and the second section has a second diameter and wherein the first diameter and the second diameter are equal.

3. The medical device of claim 1, wherein the material mixture comprises a shaped-memory alloy.

4. The medical device of claim 1, wherein the material mixture comprises nickel-titanium alloy and stainless steel.

5. The medical device of claim 1, wherein the first section and the second section are formed by a plurality of monolithic strands of stainless steel material and a shaped-memory alloy material.

6. The medical device of claim 1, wherein the second section extends from the first section to a proximal end.

7. The device of claim 1 wherein the treatment agent is a radioactive source, and the radioactive source is in the housing.

8. The device of claim 1 wherein at least one of the first and second sections has been heat treated to provide the different flexibilities.

9. A guide wire for delivery of a treatment agent to a site requiring treatment, the guide wire comprising:
a first section and a second section made from a same material mixture throughout the first section and the second section; wherein the material mixture
comprises a first material and a second material; and
wherein the first material has a first annealing temperature and the second material has a second annealing temperature, which is higher than the first annealing temperature; and wherein some of the first material has been annealed so that the first section has a flexibility that is greater than the flexibility of the second section.

10. The guide wire of claim 9 comprising a plurality of first filaments and a plurality of second filaments, the first filaments made from the first material and the second filaments made from the second material.

11. The guide wire of claim 9, wherein the first section and the second section have the same diameter.

12. The guide wire of claim 9, wherein the first material comprises a shaped-memory alloy.

13. The guide wire of claim 9, wherein the first material comprises a nickel-titanium alloy.

14. The guide wire of claim 9, wherein the second material comprises stainless steel.

15. The guide wire of claim 9, wherein the first annealing temperature is about 400° C.

16. The guide wire of claim 10, wherein the plurality of first filaments and the plurality of second filaments are equal in diameter and quantity.

17. The guide wire of claim 10, wherein the first filaments and the second filaments have diameters of different sizes.

18. The guide wire of claim 10, wherein the plurality of first filaments and the plurality of second filaments are different in quantity.

19. A medical device for delivering a treatment agent to a site requiring treatment, the device comprising:
a) a source housing comprising an interior cavity for receiving a treatment agent disposed therein; and
b) a cable comprising a first section and a second section, the first section being connected to the source housing and being closer to the housing than the second section, the first section being formed from a first material and the second section being formed from a second material, wherein the flexibility of the first section is greater than the flexibility of the second section due to treatment of at least one of the first and second materials.

20. The device of claim 19 wherein the treatment is heat treatment, and before the heat treatment the first material and the second material are the same.

21. The device of claim 20 wherein the first material and the second material are the same material mixture, the mixture comprising at least two different materials.

22. A method for forming a medical device for delivery of a radioactive source to a site requiring radiation treatment, the method comprising the steps of:
a) selecting a source housing comprising an interior cavity for receiving a radioactive source disposed therein and a cable comprising a first section and a second section, the first section being closer to the housing than the second section and being formed from a first material and the second section being formed from a second material, wherein the flexibility of at least one of the first and second materials is changeable by treatment; and
b) treating at least one of the first and second materials to change the flexibility of at least one of the first and second materials so that the flexibility of the first section is greater than the flexibility of the second section; and
c) before step b) or after step b), connecting the cable to the housing so that the first section is closer to the housing than is the second section.

23. The method of claim 22 wherein the treatment is heat treatment.

24. The method of claim 23 wherein the heat treatment is annealing.

25. The method of claim 22 wherein the housing has an opening and the step of connecting comprises snap fitting the first section into an opening of the source housing.

26. The method of claim 25 wherein the connecting step comprises welding the first section to the source housing.

27. A method for forming a medical device for delivery of a radioactive source to a site requiring radiation treatment, the method comprising the steps of:
a) providing a source housing for receiving a radioactive source disposed therein;
b) providing a cable comprising a first section and a second section made from the same material mixture throughout the first and second sections, wherein the material mixture comprises a plurality of first filaments and a plurality of second filaments, the plurality of first filaments made from a first material and the plurality of second filaments made from a second material, and wherein the first material has a first annealing temperature and the second material has a second annealing temperature, which is higher than the annealing temperature of the first material;
c) annealing the first section to at least the first annealing temperature; and
d) connecting the source housing to the cable before or after step c).

28. The method of claim 27, wherein the first material comprises a shaped memory alloy.

29. The method of claim 27, wherein the first annealing temperature is less than 600° C.

30. The method of claim 27, wherein the first material comprises nickel-titanium alloy.

31. A medical device for delivering a radioactive source to a site requiring treatment, the device comprising:
a) a source housing comprising an interior cavity;
b) a radioactive source in the housing cavity;
c) a cable comprising a first section and a second section, the first section being connected to the housing and being closer to the housing than the second section, and wherein both sections are formed from filaments of a shape-memory alloy and filaments of stainless steel, wherein the first section and the second section have the same diameter, and wherein the flexibility of the first section is less than that of the second section due to at least a portion of the shape-memory alloy filaments in the first section having been annealed.

32. The method of claim 22 wherein the step of treating comprises treating only the first section.

33. The method of claim 22 wherein the first and second materials are the same.
